(19) United States
(12) Patent Application Publication
(10) Pub. No.: US 2012/0035249 A1
(43) Pub. Date: Feb. 9, 2012

(54) Targeted Mucosal Treatment of Obesity and Diabetes

(75) Inventors: Joseph A. Kuhn, Dallas, TX (US); David M. Shanahan, Dallas, TX (US)

(73) Assignee: Dr. Joseph A. Kuhn, Dallas, TX (US)

(21) Appl. No.: 13/157,912

(22) Filed: Jun. 10, 2011

Related U.S. Application Data
(60) Provisional application No. 61/353,995, filed on Jun. 11, 2010.

(51) Int. Cl. A61K 31/7088; A61N 7/00; A61P 3/10; A61B 1/04; A61B 18/18; A61P 3/04; A61B 1/012; A61B 1/018

(52) U.S. Cl. 514/44 R; 600/104; 600/108

(57) ABSTRACT

A technique for treating obesity, diabetes, and other endocrinological disorders by removal of the omentum by endoscopic and oral approaches is described herein. The removal of the omentum interferes with the glucose absorption or ghrelin production thereby improving and correcting the obesity and diabetes.

### CROSS-REFERENCE TO RELATED APPLICATIONS

This patent application is a non-provisional patent application of U.S. provisional patent application 61/353,995 filed on Jun. 11, 2010 and entitled “Targeted Mucosal Treatment of Obesity and Diabetes” which is hereby incorporated by reference in its entirety.

### TECHNICAL FIELD OF THE INVENTION

The present invention relates in general to the field of managing and treating obesity, diabetes and other endocrinological disorders, and more particularly to a technique that interferes with glucose absorption or ghrelin production to improve and correct obesity and diabetes.

### STATEMENT OF FEDERALLY FUNDED RESEARCH

None.

### REFERENCE TO A SEQUENCE LISTING

None.

### BACKGROUND OF THE INVENTION

Without limiting the scope of the invention, its background is described in connection with techniques including removal of the omentum and associated visceral fat for the management and treatment of obesity and diabetes.

U.S. Pat. No. 4,826,480 issued to Diaz and Davis (1989) discloses an implantable catheter for use in delivering insulin or other therapeutic fluids is disclosed which uses a novel terminator design to prevent the growth of fibrotic tissue from obstruction the catheter. The catheter of the '480 patent takes advantage of fibrotic encapsulation by utilizing a depression in the surface of the terminator which will effectively keep fibrotic tissue spaced sufficiently away there from to allow fluid to be infused. In addition to being capable of long term implant, the catheter is implantable in the omentum, enabling the body's natural absorption of insulin to be successfully mimicked.

U.S. Pat. No. 7,384,630 issued to Hammerman (2008) teaches novel methods, tissues and compositions for increasing the pancreatic mass of a mammalian recipient including harvesting immature pancreatic tissue from a mammalian donor and transplanting said tissue into the peritoneal cavity (pouch of the omentum) of a mammalian recipient under conditions that allow the pancreatic tissue to become vascularized and mature, thereby developing a functioning chimeric, endocrine pancreas that produces at least insulin in the recipient. The Hammerman invention also includes mammalian immature pancreatic tissue adapted for transplantation into the peritoneal cavity of a mammalian recipient for increasing the pancreatic mass of the mammalian recipient as well as methods and compositions for treatment of the pancreatic tissue, recipient immunosuppression and recipient co-stimulatory blockade.

U.S. Patent Publication No. 20090163990 (Yang et al. 2009) describes methods for defatting omentum and processes for preparing a cellular omentum i.e., devitalized or decellularized omentum, comprising extracellular matrix for implantation into a mammalian system. Also constructs for medical applications comprising decellularized omentum.

U.S. Patent Publication No. 20090191127 (Saini, 2009) discloses mammalian omentum and methods of using the same for treating symptoms and/or conditions related to dementia are provided. In addition, a composition comprising the omentum tissue and/or the extract thereof is described in some embodiments. In some other embodiments, methods of formulating and administering the composition are also provided. In addition, a cultured cell system to culture cells and/or tissues of the omentum is described. Some aspects of embodiments provide methods of identifying one or more biological agent from the omentum tissue. In another aspect of the embodiments, methods of testing the effect of stimulation omentum in treating dementia conditions are described.

### SUMMARY OF THE INVENTION

The present invention describes a technique for treating obesity and diabetes by removal of the omentum by endoscopic and oral approaches. The removal of the omentum interferes with the glucose absorption or ghrelin production thereby improving and correcting the obesity and diabetes.

The present invention provides a device for treating or correcting one or more endocrinological disorders in a patient. The device includes an endoscope or a surgical instrument capable of accessing or being inserted into one or more natural openings or portals in the body of the patient, wherein the endoscope or the surgical instrument comprises one or more apertures or access ports; an energy source contained within the endoscope or the surgical instrument or present externally and connected to the endoscope or the surgical instrument by one or more wires connected to the apertures or the access ports, wherein the source emits a microwave energy, an acoustic energy, a laser energy, an infrared energy, an ionizing energy, an ultraviolet energy or combinations thereof; and a camera for visualizing a path of the endoscope or the surgical instrument and for focusing the energy on one or more regions of interest within the body of the patient, wherein the camera is contained within the endoscope or the surgical instrument or is present externally and is connected to the endoscope or the surgical instrument by one or more wires connected to the apertures or the access ports.

The present invention also includes a device for treating or correcting obesity, diabetes or both in a patient. The device includes a modified endoscope inserted through one or more natural openings or portals in the body of the patient, wherein the endoscope is modified to provide a microwave energy or a laser energy to one or more regions of interest in the stomach of the patient; and a camera for visualizing a path of the modified endoscope or the surgical instrument and for focusing the microwave or the laser energy on one or more regions in the stomach of the patient, wherein the camera is contained within the modified endoscope or is present externally and is connected to the modified endoscope by one or more wires connected to one or more apertures or the access ports of the endoscope.

The present invention provides a method of treating or correcting one or more endocrinological disorders selected from the group consisting of obesity, diabetes or both in a patient by identifying the patient in need of treatment against the obesity, the diabetes or both; and administering a composition comprising a therapeutically effective amount of one or more RNA interference agents dissolved or dispersed in a suitable carrier or a delivery vehicle and one or more optional pharmaceutically acceptable excipients selected from the group consisting of preservatives, antiadherents, lubricants, flavoring agents, diluents, bulking agents, stabilizers, humectants, and other organoleptic agents.

The present invention also provides a pharmaceutical composition for the treatment of one or more endocrinological disorders. The composition includes a therapeutically effective amount of one or more RNA interference agents dissolved or dispersed in a suitable carrier or a delivery vehicle; and one or more optional pharmaceutically acceptable excipients selected from the group consisting of preservatives, antiadherents, lubricants, flavoring agents, diluents, bulking agents, stabilizers, humectants, and other organoleptic agents.

The present invention includes a method of treating or correcting one or more endocrinological disorders selected from the group consisting of obesity, diabetes or both in a patient by identifying the patient in need of treatment against the obesity, the diabetes or both; inserting a modified endoscope inserted through one or more natural openings or portals in the body of the patient, wherein the endoscope is modified to provide a microwave energy or a laser energy to one or more regions of interest in the stomach of the patient; visualizing a path of the modified endoscope through the stomach of the patient using a camera, wherein the camera is contained within the modified endoscope or is present externally and is connected to the modified endoscope by one or more wires connected to one or more apertures or the access ports of the endoscope; and focusing the microwave energy or the laser energy to one or more regions of interest in the stomach to ablate, destroy or inhibit one or more glucose absorption cells, ghrelin producing cells, and cells involved in the production of hormones and peptides controlling hunger autoregulation, wherein the ablation, the destruction or the inhibition results in the treatment or the correction of the obesity, the diabetes or both in the patient.

### BRIEF DESCRIPTION OF THE DRAWINGS

None.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “obesity” as used herein implies an excess of adipose tissue. Obesity is best viewed as any degree of excess adiposity that imparts a health risk. The cutoff between normal and obese individuals can only be approximated, but the health risk imparted by the obesity is probably a continuum with increasing adiposity. The Framingham study demonstrated that a 20% excess over desirable weight clearly imparted a health risk. (Mann G V N. Engl. J. Med 291:226, 1974). In the United States a National Institutes of Health consensus panel on obesity agreed that a 20% increase in relative weight or a body mass index (BMI=body weight in kilograms divided by height in meters) above the 85th percentile for young adults constitutes a health risk. By the use of these criteria 20 to 30 percent of adult men and 30 to 40 percent of adult women in the United States are obese. (NIH, Ann Intern Med 103:147, 1985).

As used herein, the term “diabetes” is intended to include all diabetic conditions, including, without limitation, diabetes mellitus, genetic diabetes, type I diabetes, type II diabetes, and gestational diabetes. The term “diabetes” also refers to the chronic disease characterized by relative or absolute deficiency of insulin that results in glucose intolerance. Type I diabetes is also referred to as insulin dependent diabetes mellitus (IDDM) and also includes, for example, juvenile-onset diabetes mellitus. Type I is primarily due to the destruction of pancreatic β-cells. Type II diabetes mellitus is also known as non-insulin dependent diabetes mellitus (NIDDM) and is characterized, in part, by impaired insulin release following a meal. Insulin resistance can also be a factor leading to the occurrence of type II diabetes mellitus. Genetic diabetes is due to mutations which interfere with the function and regulation of β-cells. “Diabetes”, as used herein, is characterized as a fasting level of blood glucose greater than or equal to about 130 mg/dl or as a plasma glucose level greater than or equal to about 180 mg/dl as assessed at about 2 hours following the oral administration of a glucose load of about 75 g or following a meal. The term “diabetes” is also intended to include those individuals with hyperglycemia, including chronic hyperglycemia, impaired glucose homeostasis or tolerance, and insulin resistance.

The term “omentum” as used herein includes the “greater” and the “lesser” omentum. The greater omentum is a large fold of peritoneum that hangs down from the stomach, and extends from the stomach to the posterior abdominal wall after associating with the transverse colon and is associated with fat deposition, immune contribution, and wound and infection isolation. The lesser omentum is the double layer of peritoneum that extends from the liver to the lesser curvature of the stomach and the start of the duodenum.

As used herein, the term “receptor” includes, for example, molecules that reside on the surface of cells and mediate activation of the cells by activating ligands, but also is used generically to mean any molecule that binds specifically to a counterpart. One member of a specific binding pair would arbitrarily be called a “receptor” and the other a “ligand”. No particular physiological function need be associated with this specific binding. Thus, for example, a “receptor” might include antibodies, immunologically reactive portions of antibodies, molecules that are designed to complement other molecules, and so forth. Indeed, in the context of the present invention, the distinction between “receptor” and “ligand” is entirely irrelevant; the invention concerns pairs of molecules which specifically bind each other with greater affinity than either binds other molecules. However, for ease of explanation, the invention method will be discussed in terms of target receptor (again, simply a molecule for which a counterpart is sought that will react or bind with it) and “ligand” simply represents that counterpart.

The term “hormone” as used in the specification and claims refers to an organic compound formed by secretion by an endocrine gland into the circulating fluid or a synthetic analogue or derivative manufactured in vitro or in vivo.

As used herein the term “peptide” refers to two or more amino acids joined to each other by peptide bonds or modified peptide bonds. Peptides include those modified either by natural processes, such as processing and other post-translational modifications, but also chemical modification techniques. The modifications can occur anywhere in a peptide, including the peptide backbone, the amino acid side chain, and the amino or carboxyl terminal. Examples of modifications include but are not limited to amidation, acylation, acetylation, cross linking, cyclization, glycosylation, hydroxylation, phosphorylation, racemization, and covalent attachment of various moieties such as nucleotide or nucleotide derivative, lipid or lipid derivatives (see, for instance, Proteins—Structure and Molecular Properties, 2 nd Ed Creighton, W. H. Freeman and Company, New York (1993) and Post-translation covalent Modification of Proteins, B. C. Johnson, Ed., Academic Press, New York (1983)).

As used herein, the term “RNA interference (RNAi) agents” refers to gene silencing mechanisms that involving small RNAs (including miRNA and siRNA) and are frequently referred to under the broad term RNAi agents. Natural functions of RNAi include protection of the genome against invasion by mobile genetic elements such as transposons and viruses, and regulation of gene expression. “Action of RNAi agents results in the inactivation or suppression of expression of a gene within an organism.

The term “microwave energy” as used herein is refers to electromagnetic radiation that has frequencies in the range of 0.3-300 GHz. The term “infrared energy” refers to electromagnetic radiation that has a wavelength in the general range of 1-15 microns and more specifically to radiation that falls within the range 3-5 microns. The term “ultraviolet energy” refers to electromagnetic radiations that have a wave length between 100 and 400 nm (nanometers).

The terms “laser energy”, “laser radiation”, “laser beam” and variants thereof as used in the specification and in the claims encompasses a broad range of radiation frequencies, characteristics, and energy densities. The laser radiation may be suitably produced by a conventional laser such as, for example, a laser of the neodymium:yttrium aluminum garnet (Nd:YAG) type. Other laser types may include carbon dioxide, argon, holmium:yttrium aluminum garnet (holmium:YAG), and excimer. Radiation that may be used in various applications can include infrared radiation (IR), ultraviolet radiation (UV), and visible light. The term “laser ablation” as used herein refers to the removal of the target material by the focused energy of the laser beam.

As used herein, the term “treatment” refers to the treatment of the conditions mentioned herein, particularly in a patient who demonstrates symptoms of the disease or disorder.

As used herein, the term “treatment” or “treating” refers to any administration of a compound of the present invention and includes (i) inhibiting the disease in an animal that is experiencing or displaying the pathology or symptomatology of the diseased (i.e., arresting further development of the pathology and/or symptomatology) or (ii) ameliorating the disease in an animal that is experiencing or displaying the pathology or symptomatology of the diseased (i.e., reversing the pathology and/or symptomatology). The term “controlling” includes preventing treating, eradicating, ameliorating or otherwise reducing the severity of the condition being controlled.

The terms “effective amount” or “therapeutically effective amount” described herein refers to the amount of the subject compound that will elicit the biological or medical response of a tissue, system, animal or human that is being sought by the researcher, veterinarian, medical doctor or other clinician.

The terms “administration of” or “administering a” compound as used herein refers to providing a compound of the invention to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories.

The term “ghrelin” as used herein is an endogenous ligand for the growth hormone secretagogue receptor (GHSR). It is synthesized primarily in the stomach and found in the circulation of healthy humans. Ghrelin is a 28 amino acid peptide hormone with an octanoyl side chain at the third amino acid of its N-terminus (serine 3). This modification is required for the interaction at the GHS receptor and its activity. Ghrelin levels in plasma are influenced by nutritional status and are believed to regulate growth hormone (GH), appetite and fat deposition (Hataya et al. (2001) J. Clin. Endocrinol. Metab. 86: 4552; Nakazato et al. (2001) Nature 409: 194-198; Peino et al. (2000) Eur. J. Endocrinol. 143: R11-R14; Tschop et al. (2000) Nature 407: 908-913; Wren et al. (2001) Diabetes 50: 2540-2547).

As used herein the term “endoscopic” or “endoscopic surgery” are used herein to refer to all minimally invasive surgical procedures and associated instruments used to access internal surgical sites through natural openings or other portals in a body (human or otherwise). Such procedures may include arthroscopy, laparoscopy, hysteroscopy, etc. While a scope used in these procedures may simply have an eyecup at the proximal end to enable direct observation by a surgeon, preferably the proximal end of the scope is adapted to interface with an image forming device such as a video camera to enable the procedure to be viewed on a monitor. The term “laser ablation” refers to the evaporation or removal of the target material by the focused energy of a laser beam.

As used herein the term “intravenous administration” includes injection and other modes of intravenous administration. The term “subcutaneous administration” refers to administration or injection beneath the skin. Subcutaneous is also called hypodermatic, hypodermic, subdermic, subintegumental, and subtegumenta. The term “intramuscular administration” as used herein, is a broad term and is used in its ordinary sense, including, without limitation, within the substance of a muscle. The term “intraperitoneal administration” as used herein, is a broad term and is used in its ordinary sense, including, without limitation, within the peritoneal cavity, which is the area that contains the abdominal organs.

The term “pharmaceutically acceptable” as used herein to describe a carrier, diluent or excipient must be compatible with the other ingredients of the formulation and not deleterious to the recipient thereof.

The present invention discloses a technique to directly influence the mucosa of the stomach and potentially interfere with glucose absorption or ghrelin production, thereby improving or correcting obesity and diabetes. Morbid obesity is a major causative condition for the development of Type II diabetes mellitus. Rapid diabetes improvement and even resolution has been observed in gastric by-pass and gastric sleeve procedures, both of which physiologically occlude the greater omentum from the passing nutrients. The greater omentum is a poorly understood organ that's role in glucose metabolism is proposed to be temporary storage of absorbed carbohydrates for delayed transmission to the liver for inter-meal glucose supply. The use of the omentum separation technique in conjunction with the laparoscopic band procedure may provide benefits similar to those of gastric bypass and the gastric sleeve procedure in controlling Type II diabetes mellitus.

Obesity is largely caused by an excess calorie intake, which is largely driven by receptors that sense an empty stomach and signal an increased appetite through neurotransmitters which signal hunger. Eating and appetite are largely designed as automated systems to ensure nutrient intake, much like drinking is an automated process that maintains hydration through neurotransmitters which influence thirst.

The stomach is an important participant in the metabolic process of hunger and satiety. The stomach is largely a “holding tank” to allow ingestion of a large amount of food, mechanical mixing, and gradual emptying. The stomach absorbs sugar and alcohol into the cells of the stomach. Protein and fats are absorbed through enzyme systems in the small bowel. The sugars from the stomach cells are sent to the liver and the omentum where the sugars are converted to glycogen, which is a short storage form of glucose, allowing for release of additional energy to the cells in the 8-12 hours after a meal. Following a typical 2000 calorie meal, the body will absorb and convert to glucose approximately 800 calories for immediate energy requirements. The surplus 1200 calories are stored in the omentum and liver as glycogen. With obesity, the omentum becomes progressively more engorged with fat cells.

Hunger is not specifically tied to the number of calories which are ingested or the built-up reserves in the form of fat which the body retains. Rather, it is influenced by neurotransmitters that arise in the stomach and small bowel. With various stimulants (empty stomach or certain foods), the cells from the stomach secrete a neurotransmitter called ghrelin, which flows into the bloodstream and affects a certain part of the brain to stimulate hunger. There were over 1000 citations for ghrelin in scientific literature in 2009, providing excellent insight into the important role in hunger stimulation and metabolism (impact on diabetes). With weight loss surgery that removes the outer stomach (sleeve gastrectomy), the ghrelin production is permanently decreased, leading to dramatic weight reduction and offering practical evidence of the role of this hormone in obesity. The other hormones which have been identified to potentially influence hunger are listed in Example I.

The role of the omentum in energy storage and glucose metabolism is poorly understood. In patients with morbid obesity, the omentum has been shown to deliver inflammatory cytokines to the liver (C-reactive protein, tumor necrosis factor alpha, and IL-6)¹. These inflammatory cytokines potentially contribute to the metabolic syndrome, to non-alcoholic steatohepatitis, and to the persistence of glucose intolerance¹⁻³. The adipose cells are also primary sources of leptin and resistini. These factors can influence insulin resistance, TNF production, and can also lower the levels of adiponectin, which is also produced in the visceral fat of the omentum²⁻³.

The removal of the omentum from the food supply is immediately achieved by the gastric bypass and the gastric sleeve procedures. In these procedures, the resolution of diabetes is almost immediate, with most patients being discharged home off of diabetic medications. The hormonal effects of a sleeve operation are similar in some ways to the separation of the stomach and the omentum. Recent studies have shown a decrease in ghrelin and resistin, with improvement in the insulin resistance and significant improvement in diabetes². The separation of the gastro-omental blood supply theoretically could provide for immediate improvement in the metabolic syndrome. This vascular separation could also decrease the inflammatory cytokines that are released from the omentum into the portal circulation.

Studies conducted by the present inventors on omentum separation revealed a meaningful impact on glucose metabolism, based on a correction or improvement in the most basic test for diabetes (the glucose tolerance curve). After a glucose load (75 gm), the body absorbs the glucose and manages the excess. The two hour glucose level represents a standard for diabetes and pre-diabetes when this level is greater than 140 mg/dl. Following an omentum separation, the inventors found correction or significant improvement in most of the patients with early diabetes. Therefore, this experience seems to identify a novel new operative procedure to improve glucose metabolism and weight loss.

The results of the omentum separation can lead to a strategic planning for potential endoscopic approaches to further simplify the procedure and to address the mucosal interface of glucose absorption.

Methods: 1. Endoscopic Approach: (i) Endoscopic ablation via microwave energy specifically tuned to cellular components that influence glucose absorption or ghrelin production and (ii) Endoscopic ablation via laser focused on cellular location for glucose absorption or ghrelin production. 2. Oral Approach: oral delivery of targeted nanoparticles with RNA interference of protein secretion of ghrelin production

Endoscopic Microwave Energy: The microwave energy is administered with an existing endoscopic device allowing targeted energy tuned to the di-electric properties of the glucose secreting cells located in the fundus or greater curvature of the stomach. The heat generated is focused through device-specific modifications. Potential cellular targeting was optimized experimentally by involving different techniques to stimulate cellular function of ghrelin production or glucose absorption (high glucose meal, fasting, or acid reduction agents).

Endoscopic Laser Ablation: The endoscopic laser ablation of small blood vessels was safely performed in patients with diffuse bleeding over the past 15 years. The most common laser utilized was the YAG laser, which is readily available. The flexible laser wire was guided through the small access port in a standard endoscope and focused on the area of interest. The targeted mucosal ablation of glucose absorption cells or ghrelin-producing cells was directed by anatomic awareness of these cells. Specific energy limitations to optimize safety and targeting ability are required. Additional targeting ability through the use of topical fluorescein or other agents would potentially offer additional safety and specificity benefits.

Oral Delivery of RNA Interference Agent: (i) All of the 60,000 human cellular proteins are coded by DNA in the nucleus of the cell. When signaled, the DNA is turned into RNA, which is simply a specific sequence of nucleotides which provide the specific code to call into alignment one of the 26 amino acids, which are the components of all proteins. Ghrelin is a protein created in the stomach cells through this DNA to RNA to protein process. The RNA interference technology is the most specific mechanism for directly influencing the formation of Ghrelin from the stomach and thereby influencing hunger, obesity, and insulin metabolism. RNA interference relies on the awareness that a small strand of nucleotides can be constructed in such a way to block the transcription of DNA into RNA. This technology has enormous potential in the treatment of cancer and other genetic diseases. The ability to block the formation of Ghrelin offers an incredible opportunity to block hunger and manage obesity and diabetes. The DNA sequence of Ghrelin is known providing the ability to create a sequence for RNA interference is possible through collaborations and licensing agreements. The delivery vehicle for the RNA interference tool is available to initiate clinical testing with rapid clinical trials and outcomes, measured readily through serologic blood levels.

The present inventors performed a pilot study to determine the impact and safety of omentum separation on diabetes and glucose tolerance curves. The American Diabetes Association defines diabetes as having a blood glucose of 200 mg/dl or greater at the 2-hr interval of an oral glucose tolerance test (OGTT). Pre-diabetes is defined as a blood glucose value of 140-199 mg/dl at the 2-hr interval of an OGTT.

Lap band patients that were involved in the study performed an OGTT prior to their operation to determine if they are considered normal (<140 mg/dl), pre-diabetic (140-199 mg/dl), or diabetic (200 mg/dl or above).

The study conducted in the instant invention utilized 66 patients (15 men, 51 women) who had an omentum separation in addition to their lap band procedure and 45 patients (8 men, 37 women) who had no omentum separation with their lap band procedure. The average age for those who had an omentum separation was 51 years old and for those without an omentum separation the average age was 50 years old.

Out of the 66 patients that received an omentum separation, 15 (23%) of them were on diabetic medication(s) pre-operatively. At a 6-month follow up of the 15 patients on diabetic medication(s), 6 (40%) of them reported being off of all diabetic medications. Out of the 45 patients that elected to not have an omentum separation, 12 (27%) were on diabetic medication(s) preoperatively. At a 6-month follow up of the 12 patients on diabetic medication(s), 1 (8%) of them reported being off all diabetic medications.

At the 6-month follow up there was no remarkable difference between the body mass index, weight, excess body weight, or percent excess body weight of the band patients that had an omentum separation versus those who did not have an omentum separation.

A comparison of preoperative and 1-month post operative oral glucose tolerance test results for the patients with omentum separation and the patients without omentum separation was performed. Out of the patients with omentum separation, 24 participated. Out of the 24 patients, 11 (46%) of them experienced a 10 mg/dl or greater improvement in blood glucose at the 2-hour interval of the test. Four omentum separation patients with a preoperative blood glucose level above 140 mg/dl at the 2-hour interval observed a 2-hr blood glucose level below 140 mg/dl at their 1-month post operative oral glucose tolerance test. For the patients without omentum separation, 5 participated. Out of the 5 patients, 2 (40%) saw a 2-hour blood glucose improvement of at least 10 mg/dl from their preoperative to their 1-month post operative follow up. Two patients without omentum separation that had a 2-hr blood glucose level above 140 mg/dl preoperatively observed a 2-hr blood glucose level below 140 mg/dl at the 1-month post operative oral glucose tolerance test.

### EXAMPLE I

### Hormones Involved with Hunger Autoregulation

An understanding of the hormones which inhibit appetite offer possible explanations for the effectiveness of existing procedures which are used for weight loss. These hormones might serve as surrogate markers for procedures and agents which are under investigation for potential appetite suppression.

I. One important feedback mechanism to decrease appetite following a meal is related to the delivery of nutrients to the small intestine, which stimulates an increase in Glucagon-like peptide-1 (GLP-1). This hormone is secreted from the L-cells in the small intestine and has been shown experimentally to be one of the potential reasons that appetite is suppressed after gastric bypass (Korner SOARD 2007). In addition, the use of duodenal stimulation has shown increased bowel transit and improved diabetes control (glucose tolerance curves) with a proposed mechanism of action related to increased GLP-1 (Khawaled SOARD, 5: 692). Existing intravenous GLP-1 analogue medications have been shown to induce weight loss and improve diabetes in human studies (DeFronzo Diabetes Care 2005).

II. A similar enterohormone, Peptide YY (PYY) is also secreted from the L-cells and has nearly identical effects on appetite and glucose control. It has been shown to decrease appetite and food intake in human trials (Batterham NEJM 2003). Following gastric bypass or sleeve gastrectomy, there is an increase in fasting and postprandial PYY levels, offering some evidence for the decreased appetite seen with these procedures (Karamanakos Ann Surg 2008).

III. Oxyntomodulin (OXM) is a gut hormone secreted from the L-cells of the small intestine. OXM acts synergistically with PYY and GLP-1 to decrease appetite after nutrients reach the small intestine (Cummings J Clin Invest 2007). Intravenous OXM in humans decreased hunger and food intake without causing nausea (Cohen J Clin Endorcrinol Metab 2003).

IV. Pancreatic Polypeptide (PP) is a hormone released from the pancreas in response to ingestion of food. An infusion of PP can lead to decreased appetite and food intake (Batterham J Clin Endorenol Metab 2003). There are no changes in PP levels with gastric bypass or sleeve gastrectomy.

V. Cholecystokinin (CCK) is secreted by I cells located in the mucosa of the duodenum, jejunum and proximal ileum in response to a meal. It is involved with gallbladder contraction, gastric emptying, intestinal motility, and appetite suppression (Chandra Curr Oin Enocrin Diab Obes 2007; Kellum Ann Surg 1990)

The most thoroughly studied appetite stimulating hormone is Ghrelin, which is produced from the fundus of the stomach and the proximal intestine. Intravenous administration leads to increased food intake (Wren J Clin Endocrinol Metab 2001). Normal fluctuations of Ghrelin throughout the day reveals increased levels during fasting and before meals and suppression after meals. Most studies have shown a decrease in ghrelin following gastric bypass (Cummings NEJM 2002, Geloneze Obes Surg 2003). Recent studies have shown a more pronounced reduction on ghrelin after a sleeve gastrectomy Karamanakos Ann Surg 2008).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Pat. No. 4,826,480: Omentum Diffusion Catheter.

U.S. Pat. No. 7,384,630: Chimeric Pancreas.

U.S. Patent Publication No. 20090163990: Decellularized Omentum Matrix and Uses Thereof.

U.S. Patent Publication No. 20090191127: Omentum and Use Thereof.

1. Saijo Y, Kiyota N, Kawasaki Y, Miyazaki Y, Kashimura J, Fukuda M, Kishi R. Relationship between C-reactive protein and visceral adipose tissue in healthy Japanese subjects. Diabetes Obes Metab 2004; 6:249-58.

2. Bastard J P, Maachi M, Lagathu C, Kim M J, Caron M, Vidal H, Capeau J, Feve B. Recent advances in the relationship between obesity, inflammation, and insulin resistance. Eur Ctyokine Netw 2006; 17:4-12.

3. Greenfield J R, Campbell L V. Relationship between inflammation, insulin resistance and type 2 diabetes: ‘cause or effect’? Curr Diabetes Rev 2006; 2:195-211.

4. Vazquez-Vela M E, Torres N, Tovar A R. White adipose tissue as endocrine organ and its role in obesity. Arch Med Res 2008; 39:715-28.

5. Meier U, Gressner A M. Endocrine regulation of energy metabolism: review of pathobiochemical and clinical chemical aspects of leptin, ghrelin, adiponectin, and resistin. Clin Chem 2004; 50:1511-25.

6. Guerre-Millo M. Adipose tissue and adipokines: for better or worse. Diabetes Metab 2004; 30:13-9.

7. Dyck D J, Heigenhauser G J, Bruce C R. The role of adipokines as regulators of skeletal muscle fatty acid metabolism and insulin sensitivity. Acta Physiol 2006; 186:5-16.

8. Santoro S, Milleo F O, Malzoni C E, Klajner S, Borges PC, Santo M A, Campos F G, Artoni R F. Enterohormonal changes after digestive adaptation: five-year results of a surgical proposal to treat obesity and associated diseases. Obes Surg 208; 18:17-26.

What is claimed is:

1. A device for treating or correcting one or more endocrinological disorders in a patient comprising:
an endoscope or a surgical instrument capable of accessing or being inserted into one or more natural openings or portals in the body of the patient, wherein the endoscope or the surgical instrument comprises one or more apertures or access ports;
an energy source contained within the endoscope or the surgical instrument or present externally and connected to the endoscope or the surgical instrument by one or more wires connected to the apertures or the access ports, wherein the source emits a microwave energy, an acoustic energy, a laser energy, an infrared energy, an ionizing energy, an ultraviolet energy or combinations thereof; and
a camera for visualizing a path of the endoscope or the surgical instrument and for focusing the energy on one or more regions of interest within the body of the patient, wherein the camera is contained within the endoscope or the surgical instrument or is present externally and is connected to the endoscope or the surgical instrument by one or more wires connected to the apertures or the access ports.

2. The device of claim 1, wherein the endocrinological disorder is obesity, diabetes or both.

3. The device of claim 1, wherein the energy is used to ablate, destroy or inhibit one or more glucose absorption cells, ghrelin producing cells, one or more cells involved in a production of hormones and peptides controlling hunger autoregulation or combinations thereof.

4. The device of claim 3, wherein the hormones and peptides controlling hunger autoregulation are selected from the group consisting of glucagon-like-peptide 1 (GLP-1), peptide YY (PYY), oxyntomodulin (OXM), pancreatic polypeptide (PP), and cholecystokinin (CCK).

5. A device for treating or correcting obesity, diabetes or both in a patient comprising:
a modified endoscope inserted through one or more natural openings or portals in the body of the patient, wherein the endoscope is modified to provide a microwave energy or a laser energy to one or more regions of interest in the stomach of the patient; and
a camera for visualizing a path of the modified endoscope or the surgical instrument and for focusing the microwave or the laser energy on one or more regions in the stomach of the patient, wherein the camera is contained within the modified endoscope or is present externally and is connected to the modified endoscope by one or more wires connected to one or more apertures or the access ports of the endoscope.

6. The device of claim 5, wherein the energy is used to ablate, destroy or inhibit one or more glucose absorption cells, ghrelin producing cells, one or more cells involved in the production of hormones and peptides controlling hunger autoregulation or combinations thereof in the stomach of the patient.

7. The device of claim 6, wherein the hormones and peptides controlling hunger autoregulation are selected from the group consisting of glucagon-like-peptide 1 (GLP-1), peptide YY (PYY), oxyntomodulin (OXM), pancreatic polypeptide (PP), and cholecystokinin (CCK).

8. The device of claim 5, wherein the laser energy is provided by a laser wire guided through one or more small access ports in the modified endoscope.

9. A method of treating or correcting one or more endocrinological disorders selected from the group consisting of obesity, diabetes or both in a patient comprising the steps of:
identifying the patient in need of treatment against the obesity, the diabetes or both;
inserting a modified endoscope inserted through one or more natural openings or portals in the body of the patient, wherein the endoscope is modified to provide a microwave energy or a laser energy to one or more regions of interest in the stomach of the patient;
visualizing a path of the modified endoscope through the stomach of the patient using a camera, wherein the camera is contained within the modified endoscope or is present externally and is connected to the modified endoscope by one or more wires connected to one or more apertures or the access ports of the endoscope; and
focusing the microwave energy or the laser energy to one or more regions of interest in the stomach to ablate, destroy or inhibit one or more glucose absorption cells, ghrelin producing cells, and cells involved in the production of hormones and peptides controlling hunger autoregulation, wherein the ablation, the destruction or the inhibition results in the treatment or the correction of the obesity, the diabetes or both in the patient.

10. The method of claim 9, wherein the hormones and peptides controlling hunger autoregulation are selected from the group consisting of glucagon-like-peptide 1 (GLP-1), peptide YY (PYY), oxyntomodulin (OXM), pancreatic polypeptide (PP), and cholecystokinin (CCK).

11. The method of claim 9, wherein the laser energy is provided by a laser wire guided through one or more small access ports in the modified endoscope.

12. A pharmaceutical composition for the treatment of one or more endocrinological disorders comprising:
a therapeutically effective amount of one or more RNA interference agents dissolved or dispersed in a suitable carrier or a delivery vehicle; and
one or more optional pharmaceutically acceptable excipients selected from the group consisting of preservatives, antiadherents, lubricants, flavoring agents, diluents, bulking agents, stabilizers, humectants, and other organoleptic agents.

13. The composition of claim 12, wherein the RNA interference agents inhibit the formation of ghrelin and other hormones and peptides controlling hunger autoregulation by interfering with a DNA to RNA conversion step during the formation of ghrelin and the other hormones and peptides.

14. The composition of claim 13, wherein the hormones and peptides controlling hunger autoregulation are selected from the group consisting of glucagon-like-peptide 1 (GLP-1), peptide YY (PYY), oxyntomodulin (OXM), pancreatic polypeptide (PP), and cholecystokinin (CCK).

15. The composition of claim 12, wherein the composition is administered intravenously, subcutaneously, intraperitoneally, intramuscularly or orally.

16. The composition of claim 12, wherein the endocrinological disorder is obesity, diabetes or both.

17. A method of treating or correcting one or more endocrinological disorders selected from the group consisting of obesity, diabetes or both in a patient comprising the steps of:
identifying the patient in need of treatment against the obesity, the diabetes or both; and
administering a composition comprising a therapeutically effective amount of one or more RNA interference agents dissolved or dispersed in a suitable carrier or a delivery vehicle and one or more optional pharmaceutically acceptable excipients selected from the group consisting of preservatives, antiadherents, lubricants, flavoring agents, diluents, bulking agents, stabilizers, humectants, and other organoleptic agents.

18. The method of claim 17, wherein the RNA interference agents inhibit the formation of ghrelin and other hormones and peptides controlling hunger autoregulation by interfering with a DNA to RNA conversion step during the formation of ghrelin and the other hormones and peptides.

19. The method of claim 18, wherein the hormones and peptides controlling hunger autoregulation are selected from the group consisting of glucagon-like-peptide 1 (GLP-1), peptide YY (PYY), oxyntomodulin (OXM), pancreatic polypeptide (PP), and cholecystokinin (CCK).

20. The method of claim 17, wherein the composition is administered intravenously, subcutaneously, intraperitoneally, intramuscularly or orally.
